(19) United States
(12) Patent Application Publication
(10) Pub. No.: US 2010/0319721 A1
(43) Pub. Date: Dec. 23, 2010

(54) COSMETIC METHOD WHICH PROVIDES AN ELONGATING EFFECT ON THE EYELASHES AND CORRESPONDING KIT BASED ON A FILM-FORMING POLYMER

(75) Inventors: Karl Pays, Saint Maurice (FR); Stephane Arditty, Ballainvilliers (FR); Olivier Raineau, Paris (FR)

Correspondence Address: OLIFF & BERRIDGE, PLC, P.O. BOX 320850, ALEXANDRIA, VA, 22320-4850, US

(73) Assignee: L'OREAL, Paris (FR)

(21) Appl. No.: 12/808,020

(22) PCT Filed: Dec. 23, 2008
(86) PCT No.: PCT/IB08/55515

§ 371 Date: Aug. 18, 2010

Related U.S. Application Data
(60) Provisional application No. 61/022,827, filed on Jan. 23, 2008.

(30) Foreign Application Priority Data
Dec. 27, 2007 (FR) 0760384

(51) Int. Cl. A45D 40/26; A61K 8/81; A61K 8/87; A61K 8/85; A61K 8/86; A61K 8/91; A61K 8/92; A61Q 1/10

(52) U.S. Cl. 132/218; 424/70.7

(57) ABSTRACT

A method for making up the eyelashes including at least the following stages: a) forming, over all or part of the eyelashes, cohesive sheaths resulting from the application of at least one layer of at least one cosmetic composition (A) including at least 15% by weight, expressed as weight of dry matter, with respect to the total weight of said composition, of at least one film-forming compound, and b) displacing the sheaths towards the upper end of the eyelashes via a translational movement along the eyelashes over a distance which is less than the length of the eyelashes.

A subject matter of the present invention is a method for making up the eyelashes which is targeted at artificially elongating the eyelashes and a corresponding makeup kit. The invention is targeted in particular at a method for making up, with a mascara, eyelashes of natural or synthetic origin.

The term “mascara” is understood to mean a composition intended to be applied to keratinous fibers. It can be a makeup composition, a cosmetic base coating composition, also known as base coat, or a composition to be applied to a cosmetic base coating composition, also known as top coat. The mascara is more particularly intended for human eyelashes but also for false eyelashes.

In practice, there exists essentially two types of mascara formulations, namely, on the one hand, mascaras comprising an aqueous continuous phase, referred to as “emulsion mascaras”, which are provided in the form of an emulsion of waxes in water, and, on the other hand, mascaras comprising a solvent or oil continuous phase, which are anhydrous or with a low content of water and/or water-soluble solvents, are referred to as “waterproof mascaras” and are formulated in the form of a dispersion of waxes in nonaqueous solvents. There exists in particular certain mascaras which are provided in the form of an emulsion of waxes in water, also described as “waterproof”. The latter compositions are characterized by the presence of at least one latex or one pseudo latex, namely of a colloidal suspension of a film-forming polymer, which confers resistance to water on the mascara.

Generally, these compositions provide a makeup effect which can be described as volumizing on the eyelashes insofar as the deposited layer of mascara at the surface of each eyelash tends to thicken them. On the other hand, this type of composition does not usually prove to be sufficiently satisfactory in meeting another frequent expectation of users in terms of making up the eyelashes, namely an effect of elongating the eyelash.

The prior art describes mascara compositions comprising fibers in order to obtain this elongating effect on the eyelashes. These fibers can add a small amount of physical length to the eyelashes when they are sufficiently rigid and visible and when they occur at the end of the eyelash. However, the gain in physical elongation obtained via such mascaras remains moderate as it is difficult to orientate the fibers in order to stack them at the end of the eyelash. Furthermore, the presence of fibers can reduce the adhesion of the mascara to the eyelashes, lengthening the time necessary for making up.

Another technical route described in the document EP 1 430 868 is the use of mascaras exhibiting a “threading” nature at ambient temperature and which are capable of forming, when applied to keratinous fibers and after drawing out using a brush, threads in the extension of the eyelashes, without use of a heat source.

However, the ability of these mascaras to form threads at ambient temperature does not simplify their use. Thus, when the mascara is withdrawn in order to be applied, it can form threads between the container in which it is present and the applicator or between the eyelashes and the applicator. Moreover, control of the length of the threads formed on the eyelashes is problematic as the threads do not spontaneously break. In addition, the latter rarely exhibit a stiffness sufficient to remain aligned in the extension of the eyelash and to make possible a lasting elongating effect.

Consequently, a need remains for a method of making up which makes it possible to elongate the eyelashes and the present invention is targeted specifically at meeting this need.

Thus, the inventors have discovered that it is possible to obtain a significant “physical” lengthening of the eyelashes by creating extensions at their ends, via a specific makeup method, this being the case without requiring the use of a stimulus such as heat.

The invention relates mainly to a method for making up the eyelashes which comprises at least the following stages:

a) forming, over all or part of the eyelashes, cohesive sheaths resulting from the application of at least one layer of at least one cosmetic composition (A) comprising at least 15% by weight, expressed as weight of dry matter, with respect to the total weight of said composition, of at least one film-forming compound, and

b) displacing the sheaths towards the upper end of the eyelashes via a translational movement along the eyelashes over a distance which is less than the length of the eyelashes.

According to one embodiment, the sheaths are displaced over a distance which is less than three-quarters of the length of the non-made-up eyelashes.

According to one embodiment, the sheaths around the eyelashes are displaced so as to provide an elongation of at least 10%, in particular of at least 20%, with respect to the original length of the non-made-up eyelashes.

According to one embodiment, the displacement of the sheaths is facilitated by bringing the latter into contact with a composition (B) capable of reducing their adhesion to the eyelashes.

According to one embodiment, the composition (B) capable of reducing the adhesion of the sheaths to the eyelashes is applied subsequently to the formation of the sheaths.

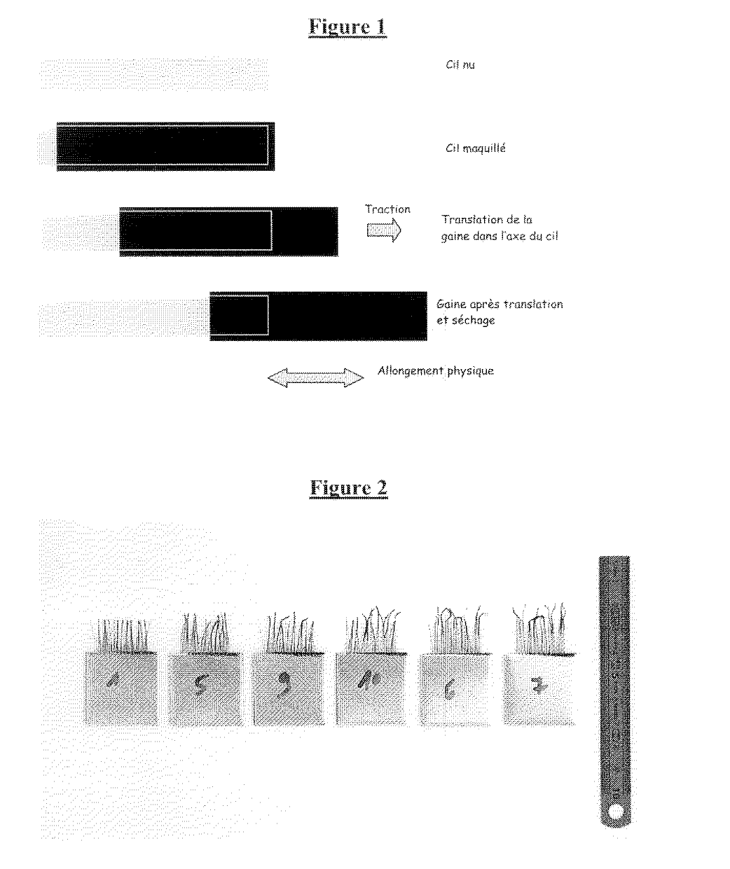

Thus, FIG. 1 diagrammatically represents the results of the various stages of the method according to the invention.

FIG. 2 is a photograph in which are represented test specimens of false eyelashes numbered 1, 5, 6, 7, 9 and 10 on a support embodying the eyelid, which gives an account of the makeup obtained on conclusion of the method according to the invention:
the set of the false eyelashes symbolized by the test specimens Nos. 1, 5, 6, 7, 9 and 10 were covered with mascara with the cosmetic composition (A) and then impregnated with water after drying the mascara individually sheathing each false eyelash,
the test specimen No. 1 was not subsequently subjected to the stage of displacement (traction/translation) of the sheaths. On the other hand, the false eyelashes symbolized by the test specimens Nos. 5, 6, 7, 9 and 10 were subjected to a stage of displacement (traction/translation) of their sheaths of mascara which are formed according to the method of the invention over a variable length.

According to another aspect, the present invention thus also relates to a method for making up the eyelashes which comprises at least the following stages:

a) applying, at the surface of the eyelashes, at least one layer of at least one cosmetic composition (A) comprising at least 15% by weight, expressed as dry matter, with respect to the total weight of said composition, of at least one film-forming compound,

b) allowing the cosmetic composition (A) applied in a) to dry in order to form sheaths around the eyelashes,

c) bringing the sheaths of composition (A), formed in stage b), into contact with pure water or an aqueous composition (B), and

d) displacing the sheaths, thus moistened, towards the upper end of the eyelashes via a translational movement along the eyelashes over a distance which is less than the length of the eyelashes.

It is understood that stage c) employs an amount of water or of aqueous solution (B) adjusted in order to make possible the moistening of the mascara sheaths and not to otherwise affect the integrity of these sheaths.

Thus, according to one embodiment, the composition (A) in stage c) is brought into contact with an amount of pure water or of aqueous composition (B) sufficient to bring about swelling of the sheath or sheaths of cosmetic composition (A).

The compositions (A) and (B) in accordance with the invention comprise a physiologically acceptable medium defined as a nontoxic medium which is capable of being applied to keratinous fibers, such as the eyelashes, and which in particular is compatible with the ocular region.

As emerges from the examples and figures presented below, the inventors have unexpectedly found that it is possible to provide an effect of elongating the eyelashes by a makeup method which consists in displacing (traction/translation), after application of mascara to the eyelashes, the resulting sheaths surrounding said eyelashes towards the upper end of the eyelash, and they have in particular found that bringing a dry deposited layer of a first composition (A) in accordance with the invention, which individually sheaths the eyelashes, into contact with a second composition (B) in accordance with the invention makes it possible to render each makeup sheath formed by the first composition (A) compatible with a longitudinal displacement. More specifically, following contact with the composition (B), each sheath becomes detached from the eyelash which it surrounds, which facilitates the stage of displacement (traction/translation) of said sheath towards the upper end of the eyelash, thus providing an elongation of the original eyelash, an artificial elongation which is set on drying the sheath thus displaced.

In addition, the inventors have found that the presence of at least one film-forming polymer in the sheaths situated around the eyelashes is favorable, during contact of the eyelashes with water or the aqueous composition (B), to swelling of these sheaths, that is to say to an increase in their volume, a phenomenon which, with regard to the makeup, advantageously generates a volumizing effect. It appears that the presence of the film-forming compound, in particular of a film-forming polymer, makes it possible to limit the evaporation of water and thus makes it possible to improve the persistence of this volumizing effect, all the more so when the water or the aqueous composition (B) are deposited subsequently to the deposition of the cosmetic composition (A) in accordance with the invention.

According to a particular embodiment, the displacement of the sheaths formed after application of the composition (A) can be performed without previous contact of the sheaths with water or any aqueous composition (B).

According to another aspect, the present invention relates to a makeup kit which can be used in the method according to the invention.

More specifically, it relates to a makeup kit comprising, in separate fashion, at least one cosmetic composition (A) comprising at least 15% by weight, expressed as dry matter, with respect to the total weight of said composition, of at least one film-forming compound and an aqueous composition (B) other than (A).

According to an alternative embodiment, the kit can comprise application means dedicated to the application of each of said compositions. For example, the kit can comprise at least one brush or one comb dedicated to the application of the composition (A).

The kit can also comprise a device favorable to the displacement of the sheaths of composition (A) towards the upper ends of the eyelashes. It can, for example, be a device of tweezers type.

According to an alternative embodiment, the kit can comprise several compositions (A) which are different from one another and which are dedicated, for example, to providing different makeups in terms of color and/or optical effect.

### DEFINITIONS ACCORDING TO THE INVENTION

The term “upper end of the eyelash” is understood to mean the end of the eyelash opposite the edge of the eyelid.

The term “displacement of the sheaths” towards the upper end of the eyelash is understood to mean a traction and a translational movement exerted on the sheaths in the direction of the end of the eyelash opposite the edge of the eyelid. The displacement of the sheaths can be carried out with the fingers or using any means favorable to said displacement, such as, for example, tweezers.

The term “volumizing effect” is understood to mean a visual perception of an effect of volume obtained in particular by coating said keratinous fibers with a cosmetic composition but which can also be provided by an optical effect due to the presence, in this cosmetic composition, of at least one material capable of generating this effect, such as, for example, a goniochromatic agent.

The term “transparent or translucent” is understood to mean the ability to allow light to pass without bringing about deviation by refraction or reflection. More particularly, the term “transparency or translucency” is understood to mean the ability to transmit, on average, at least 25% of the light in the wavelength window of 400-700 nanometers, preferably 50% of the light, through a layer of composition in accordance with the invention with a thickness of 10 microns.

Cosmetic Composition (A)

The cosmetic composition (A) can comprise an aqueous continuous phase or an oily or solvent continuous phase, in particular as defined below.

Preferably, the cosmetic composition (A) comprises an aqueous continuous phase comprising water and/or at least one water-soluble solvent.

As specified above, the cosmetic composition (A), applied to the eyelashes, forms, subsequent to the drying thereof, a coating or sheath around each eyelash. The sliding of said sheath around the eyelash is made possible by virtue of the presence in the cosmetic composition (A) of at least 15% by weight, expressed as dry matter, of at least one film-forming compound. Because of its presence under the conditions required according to the invention, the film-forming compound provides satisfactory cohesion of the sheath, thus rendering possible the sliding of the latter along the eyelash on conclusion of the contact thereof with water or the aqueous composition (B), without risk of untimely breakage.

According to another embodiment, said sliding may be obtained by the presence of a specific film forming component, under the conditions in conformity with the present invention, without necessitating the previous contact of the sheaths with water or any aqueous composition (B).

a) Film-Forming Compound

More specifically, in the present invention, the term “film-forming compound” is understood to mean a compound capable of forming, by itself alone or in the presence of an additional agent which is able to form a film, a film which is macroscopically continuous and which adheres to keratinous substances and preferably a cohesive film and better still a film having a cohesion and mechanical properties such that said film can be isolable and handleable in isolation, for example when said film is produced by casting on a nonstick surface, such as a Teflon- or silicone-treated surface.

The film-forming compound(s) is or are present in the cosmetic composition (A) in a content, as dry matter, ranging from 15% to 60% by weight, with respect to the total weight of said composition, preferably from 20% to 50% by weight and better still from 22% to 40% by weight.

Preferably, the film-forming compound is a film-forming polymer.

Mention may be made, among the film-forming polymers which can be used in the cosmetic composition (A), of the synthetic polymers defined below, of radical type or of polycondensate type, polymers of natural origin and their blends.

These film-forming polymers are preferably distinct from the polyelectrolytes defined below.

The film-forming polymer can be dispersed in the form of solid particles in an aqueous phase of the composition (A) or else be dissolved or dispersed in the form of solid particles in a liquid fatty phase of the composition (A). The composition (A) can also comprise a blend of these film-forming polymers.

The film-forming polymer can also be a polymer dissolved in an oily or solvent phase comprising oils or organic solvents, such as those described below. It is then said that the film-forming polymer is a fat-soluble film-forming polymer. The film-forming polymer can be a polymer dissolved in an aqueous phase; it is then said that it is a water-soluble film-forming polymer.

Water-Soluble Film-Forming Polymer:

Mention may in particular be made, by way of illustration of this type of polymer, of:
proteins, such as proteins of plant origin, such as, for example, wheat or soya proteins; or proteins of animal origin, such as keratins, for example keratin hydrolysates and sulfonic keratins;
cellulose polymers, such as, for example, hydroxyethylcellulose, hydroxypropylcellulose, methylcellulose or ethylhydroxyethylcellulose;
acrylic polymers or copolymers, such as, for example, polyacrylates or polymethacrylates;
vinyl polymers, such as, for example, polyvinylpyrrolidones, copolymers of methyl vinyl ether and of maleic anhydride, the copolymer of vinyl acetate and of crotonic acid, copolymers of vinylpyrrolidone and of vinyl acetate, copolymers of vinylpyrrolidone and of caprolactam, or polyvinyl alcohol;
gums arabic, guar gum, xanthan derivatives or karaya gum;
alginates and carrageenans;
glycoaminoglycans, hyaluronic acid and its derivatives;
shellac resin, gum sandarac, dammars, elemis or copals;
deoxyribonucleic acid;
muccopolysaccharides, such as chondroitin sulfates; and
their mixtures.

Fat-Soluble Film-Forming Polymer

Mention may in particular be made, by way of illustration of this type of polymer, of:
copolymers of vinyl ester (the vinyl group being directly connected to the oxygen atom of the ester group and the vinyl ester having a saturated, linear or branched, hydrocarbon radical of 1 to 19 carbon atoms bonded to the carbonyl of the ester group) and of at least one other monomer which can be a vinyl ester (other than the vinyl ester already present), an α-olefin (having from 8 to 28 carbon atoms), an alkyl vinyl ether (the alkyl group of which comprises from 2 to 18 carbon atoms) or an allyl or methallyl ester (having a saturated, linear or branched, hydrocarbon radical of 1 to 19 carbon atoms bonded to the carbonyl of the ester group).

These copolymers can be crosslinked using crosslinking agents which can be either of the vinyl type or of the allyl or methallyl type, such as tetraallyloxyethane, divinylbenzene, divinyl octanedioate, divinyl dodecanedioate and divinyl octadecanedioate.

Mention may be made, as examples of these copolymers, of the copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% of divinylbenzene, vinyl dimethylpropionate/vinyl laurate, crosslinked with 0.2% of divinylbenzene, vinyl acetate/octadecyl vinyl ether, crosslinked with 0.2% of tetraallyloxyethane, vinyl acetate/allyl stearate, crosslinked with 0.2% of divinylbenzene, vinyl acetate/1-octadecene, crosslinked with 0.2% of divinylbenzene, and allyl propionate/allyl stearate, crosslinked with 0.2% of divinylbenzene.
copolymers resulting from the copolymerization of vinyl esters having from 9 to 22 carbon atoms or of alkyl acrylates or methacrylates, the alkyl radicals having from 10 to 20 carbon atoms.

Such fat-soluble copolymers can be chosen from copolymers of poly(vinyl stearate), of poly(vinyl stearate) crosslinked using divinylbenzene, diallyl ether or diallyl phthalate, copolymers of poly(stearyl(meth)acrylate), of poly(vinyl laurate), of poly(lauryl (meth)acrylate), it being possible for these poly(meth)acrylates to be crosslinked using ethylene glycol dimethacrylate or tetraethylene glycol dimethacrylate.

The fat-soluble copolymers defined above are known and are described in particular in application FR-A-2 232 303. They can have a weight-average molecular weight ranging from 2000 to 500 000 and preferably from 4000 to 200 000.
Polyalkylenes and in particular copolymers of C₂-C₂₀ alkenes, such as polybutene, alkylcelluloses with a saturated or unsaturated and linear or branched C₁ to C₈ alkyl radical, such as ethylcellulose and propylcellulose, copolymers of vinylpyrrolidone (abbreviated to VP) and in particular copolymers of vinylpyrrolidone and of C₂ to C₄₀ alkene and better still C₃ to C₂₀ alkene. Mention may be made, as example of VP copolymer which can be used in the invention, of VP/vinyl acetate, VP/ethyl methacrylate, VP/ethyl methacrylate/methacrylic acid, VP/eicosene, VP/hexadecene, VP/triacontene, VP/styrene or VP/acrylic acid/lauryl methacrylate copolymer or butylated polyvinylpyrrolidone (PVP).
Linear block ethylenic polymers, in particular which comprise, preferably, at least one first block and at least one second block having different glass transition temperatures (Tg), said first and second blocks being connected to one another via an intermediate block comprising at least one constituent monomer of the first block and at least one constituent monomer of the second block. Advantageously, the first and second blocks of the block polymer are incompatible with one another.

Such polymers are described, for example, in the documents EP 1 411 069 or WO 04/028488.
Silicone resins, generally soluble or swellable in silicone oils, which are crosslinked polyorganosiloxane polymers. The nomenclature of silicone resins is known under the name of “MDTQ”, the resin being described according to the various siloxane monomer units which it comprises, each of the letters “MDTQ” characterizing one type of unit.

These silicone polymers can belong to the following two families:
polyorganosiloxanes comprising at least two groups capable of establishing hydrogen interactions, these two groups being situated in the chain of the polymer, and/or
polyorganosiloxanes comprising at least two groups capable of establishing hydrogen interactions, these two groups being situated on grafts or branches, and
their blends.

Mention may be made, as examples of silicone resins, of the polymethylsilsesquioxanes sold by Wacker, such as, for example, Wacker Belsil® PMS MK Powder, and by Shin-Etsu under the reference KR-220L.

Mention may also be made, as examples of silicone resins, of siloxysilicate resins, in particular trimethylsiloxysilicate (abbreviated to TMS) resins. They are sold in particular under the reference SR1000 by General Electric or under the reference TMS 803 by Wacker. Mention may also be made of trimethylsiloxysilicate resins sold in a solvent, such as cyclomethicone, sold under the names “KF-7312J” by Shin-Etsu or “DC 749” or “DC 593” by Dow Corning.

Use may also be made of silicone polyamides of the polyorganosiloxane type, such as those described in the documents U.S. Pat. No. 5,874,069, U.S. Pat. No. 5,919,441, U.S. Pat. No. 6,051,216 and U.S. Pat. No. 5,981,680.

As specified above, the film-forming polymer can also be employed in a form dispersed in the composition in accordance with the invention, that is to say that it can be present in the form of particles in dispersion in an aqueous or nonaqueous phase.

The techniques for preparing these dispersions are well known to a person skilled in the art.

Use may be made, as nonaqueous dispersions of film-forming polymer, of dispersions of particles of a grafted ethylenic polymer, preferably acrylic polymer, in a liquid oily phase:
either in the form of ethylenic polymer particles dispersed in the absence of additional stabilizer at the surface of the particles, such as described in particular in the document WO 04/055081,
or in the form of surface-stabilized particles dispersed in the liquid fatty phase. The dispersion of surface-stabilized polymer particles can be manufactured as described in the document EP-A-749 747. The polymer particles can be stabilized at the surface by virtue of a stabilizer which can be a block polymer, a grafted polymer and/or a random polymer, alone or as a blend. Dispersions of film-forming polymer in the liquid fatty phase, in the presence of stabilizing agents, are described in particular in the documents EP-A-748 746, EP-A-923 928 and EP-A-930 060, the contents of which are incorporated by way of reference in the present patent application.

Advantageously, use is made of dispersions of ethylenic polymer particles dispersed in the absence of additional stabilizer at the surface of said particles.

Mention may also be made of acrylic dispersions in isododecane, such as Mexomer PAP®, sold by Chimex.

Preferably, the material concerned is an aqueous dispersion of particles of latexes or pseudolatexes as defined below.

Latexes and pseudolatexes are colloidal dispersions of polymer particles in an aqueous liquid phase. The terms “aqueous dispersion of polymer particles” and “latexes and pseudolatexes” are used without distinction in the context of the description of the invention.

Latexes are generally obtained by suspension or emulsion polymerization or copolymerization of monomers according to processes well known to a person skilled in the art. Such monomers can in particular be chosen from styrene, butadiene, acrylonitrile, chloroprene, vinyl acetate, urethanes, isoprene, isobutylene and acrylic or methacrylic, maleic, crotonic or itaconic acids or their esters or amides.

The expression “pseudolatexes” denotes a dispersion composed of particles, generally spherical, of a polymer, these particles being obtained by dispersion of the polymer in an appropriate aqueous phase.

The expression “pseudolatex” should not be confused with the expression “latex” or “synthetic latex”, which is also a dispersion composed of particles of a polymer which are obtained directly by polymerization of one or more monomers in an appropriate aqueous phase, as mentioned above.

These latexes or pseudolatexes have advantageous film-forming properties in conferring, on the cosmetic compositions in accordance with the invention, good resistance to water. The polymers included in these latexes or pseudolatexes are thus consequently also called film-forming polymers.

Mention may more particularly be made, among the film-forming polymers which can be included in the latex or the pseudolatex under consideration according to the present invention, of synthetic polymers of the polycondensate type or of the radical type.

Mention may be made, among synthetic film-forming polymers of polycondensate type, of polyurethanes, which may be anionic, cationic, nonionic or amphoteric, polyurethane-acrylics, polyurethane-polyvinylpyrrolidones, polyester-polyurethanes, polyetherpolyurethanes, polyureas, polyurea-polyurethanes, and the blends of these.

The polyurethanes can, for example, be a copolymer of aliphatic, cycloaliphatic or aromatic polyurethane, of polyurea-polyurethane or of polyurea comprising, alone or as a mixture:
at least one block of linear or branched, aliphatic and/or cycloaliphatic and/or aromatic, polyester origin, and/or
at least one block of aliphatic and/or cycloaliphatic and/or aromatic polyether origin, and/or
at least one substituted or unsubstituted, branched or unbranched, silicone block, for example of polydimethylsiloxane or of polymethylphenylsiloxane, and/or
at least one block comprising fluorinated groups.

The polyurethanes as defined in the invention can also be obtained from branched or unbranched polyesters or from alkyds comprising mobile hydrogens which are modified by means of a polyaddition with a diisocyanate and a bifunctional organic coreactant compound (for example dihydro, diamino or hydroxyamino), additionally comprising either a carboxylate or carboxylic acid group or a sulfonate or sulfonic acid group, indeed even a neutralizable tertiary amine group or a quaternary ammonium group.

Mention may also be made of polyesters, polyesteramides, fatty-chain polyesters, polyamides and epoxy ester resins.

The polyesters can be obtained in a known way by means of the polycondensation of aliphatic or aromatic diacids with aliphatic or aromatic diols or with polyols. Succinic acid, glutaric acid, adipic acid, pimelic acid, suberic acid or sebacic acid can be used as aliphatic diacids. Terephthalic acid or isophthalic acid, indeed even a derivative, such as phthalic anhydride, can be used as aromatic diacids. Ethylene glycol, propylene glycol, diethylene glycol, neopentyl glycol, cyclohexanedimethanol and 4,4-N-(1-methylpropylidene)bisphenol can be used as aliphatic diols. Glycerol, pentaerythritol, sorbitol and trimethylolpropane can be used as polyols.

The polyesteramides can be obtained analogously to the polyesters by means of the polycondensation of diacids with diamines or aminoalcohols. Ethylenediamine, hexamethylenediamine and meta- or para-phenylenediamine can be used as diamine. Monoethanolamine can be used as aminoalcohol.

Mention may be made, as monomer carrying an anionic group which can be used during the polycondensation, for example, of dimethylolpropionic acid, trimellitic acid or a derivative, such as trimellitic anhydride, the sodium salt of the acid 3-sulfopentanediol and the sodium salt of 5-sulfo-1,3-benzenedicarboxylic acid. The polyesters having a fatty chain can be obtained via the use of diols having a fatty chain during the polycondensation. The epoxy ester resins can be obtained by the polycondensation of fatty acids with a condensate at the α,ω-diepoxy ends.

The terms “synthetic film-forming polymers of radical type” or “radical polymers” are understood to mean a polymer obtained by polymerization of monomers possessing unsaturation, in particular ethylenic unsaturation, each monomer being capable of homopolymerizing (unlike polycondensates).

The radical polymers can in particular be acrylic and/or vinyl homopolymers or copolymers.

The acrylic polymers can result from the copolymerization of monomers chosen from acrylic acid or methacrylic acid esters and/or amides.

The esters of acid monomers are advantageously chosen from esters of (meth)acrylic acid (also known as (meth)acrylates), in particular alkyl(meth)acrylates, especially C₁-C₃₀ alkyl(meth)acrylates, preferably C₁-C₂₀ alkyl(meth)acrylates, aryl (meth)acrylates, especially C₆-C₁₀ aryl(meth)acrylates, or hydroxyalkyl(meth)acrylates, especially C₂-C₆ hydroxyalkyl(meth)acrylates.

Mention may be made, as examples of monomers of the ester type, of methyl methacrylate, ethyl(meth)acrylate, butyl(meth)acrylate, isobutyl(meth)acrylate, 2-ethylhexyl(meth)acrylate, lauryl(meth)acrylate and cyclohexyl(meth)acrylate. Mention may be made, as examples of monomers of the amide type, of N-(t-butyl)acrylamide and N-(t-octyl)acrylamide.

Mention may be made, as amides of the acid monomers, for example, of (meth)acrylamides and in particular N-alkyl(meth)acrylamides, especially (C₂-C₁₂ alkyl)(meth)acrylamides. Mention may be made, among N-alkyl(meth)acrylamides, of N-ethylacrylamide, N-(t-butyl)acrylamide, N-(t-octyl)acrylamide and N-undecylacrylamide.

Use is preferably made of the acrylic polymers obtained by the copolymerization of monomers possessing ethylenic unsaturation comprising hydrophilic groups, preferably of nonionic nature, such as hydroxyethyl acrylate, 2-hydroxypropyl acrylate, hydroxyethyl methacrylate and 2-hydroxypropyl methacrylate.

The vinyl polymers can result from the homopolymerization or from the copolymerization of monomers chosen from vinyl esters, styrene or butadiene. Mention may be made, as examples of vinyl esters, of vinyl acetate, vinyl neodecanoate, vinyl pivalate, vinyl benzoate and vinyl t-butylbenzoate.

Mention may be made, as styrene monomers, of styrene and α-methylstyrene.

Use may also be made of acrylic/silicone copolymers, indeed even nitrocellulose/acrylic copolymers.

Mention may also be made of the polymers resulting from the radical polymerization of one or more radical monomers inside and/or partially at the surface of preexisting particles of at least one polymer chosen from the group consisting of polyurethanes, polyureas, polyesters, polyesteramides and alkyds. These polymers are generally known as “hybrid polymers”.

Use may be made, as aqueous dispersion of film-forming polymer, of the acrylic dispersions sold under the names “Neocryl XK-90®”, “Neocryl A-1070®”, “Neocryl A-1090®”, “Neocryl BT-62®”, “Neocryl A-1079®” and “Neocryl A-523®” by Avecia-Neoresins, “Dow Latex 432®” by Dow Chemical, “Daitosol 5000 AD®” or “Daitosol 5000 SJ®” by Daito Kasey Kogyo, “Syntran 5760®” by Interpolymer or Allianz Opt® by Röhm & Haas, or the aqueous polyurethane dispersions sold under the names “Neorez R-981®” and “Neorez R-974®” by Avecia-Neoresins, “Avalure UR405®”, “Avalure UR-410®”, “Avalure UR-425®”, “Avalure UR-450®”, “Sancure 875®”, “Sancure 861®”, “Sancure 878®” and “Sancure 2060®” by Goodrich, “Impranil 85®” by Bayer or “Aquamere H-1511®” by Hydromer, the sulfopolyesters sold under the trade name “Eastman AQ®” by Eastman Chemical Products, vinyl dispersions, such as “Mexomer PAM”, aqueous dispersions of poly(vinyl acetate), such as Vinybran® from Nisshin Chemical or those sold by Union Carbide, aqueous dispersions of vinylpyrrolidone, dimethylaminopropylmethacrylamide and lauryldimethyl-methacrylamidopropylammonium chloride terpolymer, such as Styleze W from ISP, aqueous dispersions of polyurethane-polyacrylic hybrid polymers, such as those sold under the references “Hybridur®” by Air Products or “Duromer®” by National Starch, dispersions of core/shell type: for example those sold by Arkema under the brand Kynar® (core: fluorinated-shell: acrylic) or those described in the document U.S. Pat. No. 5,188,899 (core: silica-shell: silicone), and their mixtures.

Advantageously, the material concerned is the aqueous dispersion sold under the trade name Avalure UR450®.

The polymer constituting the particles of the aqueous dispersion advantageously does not comprise an ionizable monomer. The sulfopolyesters obtained by condensation of diethylene glycol, of cyclohexanedimethanol, of isophthalic acid and of sulfoisophthalic acid comprise in particular such ionizable monomers, which confers on them a degree of affinity for water. Preference is given, on the contrary, to the compositions in which the film-forming polymers do not exhibit such an affinity for water.

Mention may in particular be made, as water-dispersible film-forming polymer of latex type very particularly suitable for the invention, of acrylate copolymers and more particularly acrylate copolymers as a 40% emulsion, such as those sold by Interpolymer under the trade name Syntran® 5760.

According to one embodiment of the invention, the composition can comprise a plasticizing agent which promotes the formation of a film with the film-forming polymer. Such a plasticizing agent can be chosen from any compound known to a person skilled in the art as being capable of performing the desired role.

For obvious reasons, the choice of the physiological acceptable medium forming the first composition (A) is directly related to the nature of the film-forming polymer selected.

In the preferred alternative form where the film-forming polymer is a polymer in dispersion in an aqueous phase or latex, the composition (A) then comprises an aqueous phase comprising water and/or at least one water-soluble solvent.

The term “water-soluble solvent” denotes, in the present invention, a compound which is liquid at ambient temperature and which is miscible with water (miscibility in water of greater than 50% by weight at 25° C. and atmospheric pressure).

Mention may in particular be made, among the water-soluble solvents which can be used in the composition (A), of lower monoalcohols having from 1 to 5 carbon atoms, such as ethanol and isopropanol, glycols having from 2 to 8 carbon atoms, such as ethylene glycol, propylene glycol, 1,3-butylene glycol and dipropylene glycol, C₃ and C₄ ketones and C₂-C₄ aldehydes.

The aqueous phase (water and/or water-soluble solvent(s)) can be introduced as such into the composition (A) or can be incorporated therein via one or more ingredients constituting said composition (A). Thus, water can in particular be introduced into the cosmetic composition (A) via the introduction of latex or pseudolatex, that is to say via the aqueous dispersion of polymer particles, or also of inverse latex, such as the various types of compounds sold under the trade name Simulgel®.

According to another preferred alternative embodiment, the composition (A) according to the invention comprises at least one polyelectrolyte.

b) Polyelectrolyte

The term “polyelectrolyte” is understood to mean, within the meaning of the invention, a macromolecular substance which has the ability to dissociate, when it is dissolved in water or in any other ionizing medium, to give at least one ion. In other words, a polyelectrolyte is a polymer comprising at least one ionizable monomer.

The presence of such an electrolyte in the composition (A) makes it possible to generate, on contact with water or with the aqueous composition (B) deposited subsequently on the made-up eyelash, a swelling effect which is advantageously expressed by a volumizing effect on the eyelashes which is particularly valued in the field of cosmetics.

A polyelectrolyte can be a polyacid, a polybase, a polysalt or a polyampholyte. In the context of the invention, it is preferably a polyacid or more advantageously still a strong polyacid.

In particular, the polyelectrolyte can give polyions, for example polyanions, when it is dissociated in water.

Preferably, the polyelectrolyte included in the cosmetic composition (A) in accordance with the invention is a branched and/or crosslinked anionic polymer.

The counterions of the polyions formed during the dissociation can be of any nature, inorganic or organic.

In particular, when the polyelectrolyte is a branched or crosslinked anionic polymer, the cations can be alkali metal or alkaline earth metal cations, such as sodium or potassium, or also the ammonium ion.

The sodium cation Na⁺ is preferred; this is why it is mainly cited in the list of polyelectrolytes which will follow, without this constituting any limitation on this specific counterion.

Mention may be made, as polyelectrolyte suitable for the invention, of:
the acrylamide/Na AMPS copolymer, such as Simulgel 600® in the form of an emulsion comprising polysorbate 80 as surfactant and comprising isohexadecane as oil phase, sold by SEPPIC, or Simulgel EG®, Simulgel A® and Simulgel 501®, which are sold by the same company. Simulgel 600® is described in particular in the document FR 2 785 801. It is in reality an inverse latex. The polyelectrolyte AMPS is 2-methyl-2-[(1-oxo-2-propenyl)amino]-1-propanesulfonic acid, partially or completely salified, in particular in the form of the sodium salt or of the ammonium salt, included at a level of 30 to 50% in molar proportions in the mixture comprising AMPS and also an acrylamide, for its part present at a level of 50 to 70%,
crosslinked sodium starch glycolate in the powder form;
sodium polyacrylates, such as Norsocryl 535®, sold by Arkema, or Cosmedia SP®, sold by Cognis,
ionizable derivatives of polysaccharides, such as cellulose salts and sodium alginates,
grafted starch-based copolymers, such as the Waterlock® products (for example A-180 and G-400) from Grain Processing Corporation,
polyacrylic acids of Synthlen K® type,
polyacrylic acid/alkyl acrylate copolymers of Pemulen® type,
AMPS (polyacrylamidomethylpropanesulfonic acid partially neutralized with ammonia and highly crosslinked), for example sold by Clariant,
copolymers of AMPS and of alkyl methacrylates which are polyoxyethylenated (and crosslinked or noncrosslinked),
sodium carboxymethylcellulose and all ionizable derivatives of cellulose, and
their mixtures.

Sodium polyacrylate and the acrylamide/AMPS copolymer and their copolymers are very particularly suitable for the invention. Preferably, the material concerned is the polyelectrolyte sold under the trade name Simulgel 600®.

When the volumizing effect is desired in the composition in accordance with the invention, it will, of course, be seen to that the content of polyelectrolyte is adjusted so that this effect can actually be generated.

It is understood that the amount of polyelectrolyte is capable of varying significantly according to the nature of the polyelectrolyte. This amount is at least equal to the amount necessary and sufficient to confer, on the cosmetic composition (A), a volumizing effect when the latter is desired. It is also described as effective amount.

Advantageously, the polyelectrolyte is present in the cosmetic composition (A) in a content ranging from 0.05 to 15%, by weight of dry matter, with respect to the total weight of the composition, more preferably still from 0.1 to 10% and better still from 0.5 to 5%.

In addition to at least one film-forming agent and, if appropriate, an additional agent which is able to form a film, the composition (A) can comprise other ingredients conventionally present in mascara compositions, such as, for example, fillers, surfactants or coloring materials. Such compounds are more particularly described below.

After it has been applied to the eyelashes, the composition (A) is dried. This drying can be carried out at ambient temperature or, if appropriate, by application of a suitable method of drying, for example of heating comb type.

This drying is carried out under favorable conditions (drying time, drying temperature) in order to make it possible to form, from the deposited layer of the first composition (A), a sheath of makeup surrounding each of the made-up eyelashes.

Composition (B)

As specified above, the eyelashes made up with the composition (A) can advantageously be brought into contact with a second aqueous composition (B) intended to favor better mobility of the sheaths of makeup film which are formed from the first composition (A) at the surface of the eyelashes.

The second composition (B) is formed in all or part of water. According to a first alternative form, it can be formed only of water. According to a second alternative form, it is an aqueous solution, that is to say is formed of water as a mixture with one or more water-soluble solvents, in particular as defined above.

Use may be made, as composition (B), of any aqueous composition known to a person skilled in the art.

If the composition (A) can be provided, for example, in the form of an emulsion, of a mascara of base coat type, the aqueous composition (B) for its part can be provided in the form of a mascara of top coat type, of a milk, of an emulsion, of a lotion.

When the aqueous composition (B) or the cosmetic composition (A) are provided in the form of an emulsion, it can be a water-in-oil (W/O) or oil-in-water (O/W) emulsion or a multiple (W/O/W or O/W/O) emulsion.

This aqueous composition (B) preferably comprises an aqueous continuous phase, if appropriate in combination with an oily phase, in particular as defined below.

This aqueous composition (B) can also comprise one or more surfactants.

For obvious reasons, the choice of these surfactants, as well as their respective amounts, are adjusted so as not to affect the effectiveness of the composition (B) with regard to the sheaths formed by the composition. Thus, the surfactants present in the composition (B) must not provide a detergent role with respect to said composition (A).

Contact of the composition (B) with the eyelashes made up by the composition (A) can be achieved manually, for example by application of a support impregnated with the composition (B) to made-up eyelashes. This support can be a brush, a comb, a substrate of wipe type or any other tool allowing it to be applied.

The duration of this contact is adjusted in order to make possible the desired moistening of the eyelashes, that is to say the moistening favorable to the improvement in the displacement of the makeup sheaths, thus better detached from the eyelash towards the upper end of the eyelashes.

This displacement can be achieved manually or using any other tool which makes this displacement possible, such as tweezers of the type of those provided for applying heating of the eyelashes. Several sheaths dispersed over consecutive eyelashes can thus be displaced simultaneously. On conclusion of the displacement of these sheaths towards the end of the eyelashes and the positioning of the makeup sheaths, the latter again set by drying, thus recovering their original level of adhesion.

Surfactants

The composition (A) and/or (B), preferably the composition (B), can comprise at least one surface-active agent chosen from nonionic, anionic, cationic or amphoteric surfactants or surface-active emulsifiers. Reference may be made to the document “Encyclopedia of Chemical Technology, Kirk-Othmer”, volume 22, pp. 333-432, 3rd edition, 1979, Wiley, for the definition of the properties and functions (emulsifying) of surfactants, in particular pp. 347-377 of this reference for anionic, amphoteric and nonionic surfactants.

Such surface-active agents can be present in particular in a proportion ranging from 0.1% to 20% by weight and better still from 0.3% to 15% by weight, with respect to the total weight of the composition.

For obvious reasons, the choice of the surfactants is made with regard to the type of formulation selected for the compositions (A) and (B), according to whether an aqueous phase or an oil-in-water or water-in-oil emulsion is concerned.

Thus, in order to obtain an oil-in-water emulsion, use may in particular be made of an emulsifying surface-active agent having, at 25° C., an HLB (hydrophilic-lipophilic balance) within the meaning of Griffin of greater than or equal to 8. The HLB value according to Griffin is defined in J. Soc. Cosm. Chem., 1954 (volume 5), pages 249-256.

The surfactants which can be used in the aqueous composition (B) in accordance with the invention can be chosen:
from nonionic surfactants:

a) nonionic surface-active agents with an HLB of greater than or equal to 8 at 25° C., used alone or as a mixture; mention may in particular be made of:
oxyethylenated and/or oxypropylenated glycerol ethers which can comprise from 1 to 150 oxyethylene and/or oxypropylene units;
oxyethylenated and/or oxypropylenated ethers (which can comprise from 1 to 150 oxyethylene and/or oxypropylene units) of fatty alcohols, in particular C₈-C₂₄ and preferably C₁₂-C₁₈ fatty alcohols, such as the oxyethylenated ether of stearyl alcohol comprising 20 oxyethylene units (CTFA name “Steareth-20”), such as Brij 78, sold by Uniqema, the oxyethylenated ether of cetearyl alcohol comprising 30 oxyethylene units (CTFA name “Ceteareth-30”) and the oxyethylenated ether of the mixture of C₁₂-C₁₅ fatty alcohols comprising 7 oxyethylene units (CTFA name “C₁₂₋₁₅ Pareth-7”), such as that sold under the name Neodol 25-7® by Shell Chemicals;
esters of a fatty acid, in particular a C₈-C₂₄ and preferably C₁₆-C₂₂ fatty acid, and of polyethylene glycol (or PEG) (which can comprise from 1 to 150 oxyethylene units), such as PEG-50 stearate and PEG-40 monostearate, sold under the name Myrj 52P® by Uniqema;
esters of a fatty acid, in particular a C₈-C₂₄ and preferably C₁₆-C₂₂ fatty acid, and of oxyethylenated and/or oxypropylenated glycerol ethers (which can comprise from 1 to 150 oxyethylene and/or oxypropylene units), such as the polyoxyethylenated glyceryl monostearate comprising 200 oxyethylene units sold under the name Simulsol 220 TM® by SEPPIC; polyoxyethylenated glyceryl stearate comprising 30 oxyethylene units, such as the product Tagat S® sold by Goldschmidt, polyoxyethylenated glyceryl oleate comprising 30 oxyethylene units, such as the product Tagat O® sold by Goldschmidt, polyoxyethylenated glyceryl cocoate comprising 30 oxyethylene units, such as the product Varionic LI 13® sold by Sherex, polyoxyethylenated glyceryl isostearate comprising 30 oxyethylene units, such as the product Tagat L® sold by Goldschmidt, and polyoxyethylenated glyceryl laurate comprising 30 oxyethylene units, such as the product Tagat I® from Goldschmidt;
esters of a fatty acid, in particular a C₈-C₂₄ and preferably C₁₆-C₂₂ fatty acid, and of oxyethylenated and/or oxypropylenated sorbitol ethers (which can comprise from 1 to 150 oxyethylene and/or oxypropylene units), such as the polysorbate 60 sold under the name Tween 60® by Uniqema;
dimethicone copolyol, such as that sold under the name Q2-5220® by Dow Corning;
dimethicone copolyol benzoate, such as that sold under the name Finsolv SLB 101® and 201® by Fintex;
copolymers of propylene oxide and of ethylene oxide, also known as EO/PO polycondensates;
and their mixtures.

The EO/PO polycondensates are more particularly copolymers consisting of polyethylene glycol and polypropylene glycol blocks, such as, for example, polyethylene glycol/polypropylene glycol/polyethylene glycol triblock polycondensates.

These triblock polycondensates have, for example, the following chemical structure:

H—(O—CH₂—CH₂)ₐ—(O—CH(CH₃)—CH₂)_{b}—(O—CH₂—CH₂)ₐ—OH

in which formula a ranges from 2 to 120 and b ranges from 1 to 100.

The EO/PO polycondensates preferably have a weight-average molecular weight ranging from 1000 to 15 000 and better still ranging from 2000 to 13 000. Advantageously, said EO/PO polycondensates have a cloud point, at 10 g/l in distilled water, of greater than or equal to 20° C., preferably of greater than or equal to 60° C. The cloud point is measured according to the ISO 1065 standard. Mention may be made, as EO/PO polycondensate which can be used according to the invention, of the polyethylene glycol/polypropylene glycol/polyethylene glycol triblock polycondensates sold under the Synperonic® names, such as Synperonic PE/L44® and Synperonic PE/F127®, by ICI.

b) nonionic surface-active agents with an HLB of less than 8 at 25° C., optionally in combination with one or more nonionic surface-active agents with an HLB of greater than 8 at 25° C., such as mentioned above, such as:
esters and ethers of monosaccharides, such as sucrose stearate, sucrose cocoate, sorbitan stearate and their mixtures, for example
Arlatone 2121®, sold by ICI, or Span 65V, from Uniqema;
esters of fatty acids, in particular C₈-C₂₄ and preferably C₁₆-C₂₂ fatty acids, and of a polyol, in particular of glycerol or of sorbitol, such as glyceryl stearate, for example sold under the name Tegin M® by Goldschmidt, glyceryl laurate, such as the product sold under the name Imwitor 312® by Hüls, polyglyceryl-2 stearate, sorbitan tristearate and glyceryl ricinoleate;
oxyethylenated and/or oxypropylenated ethers, such as the oxyethylenated ether of stearyl alcohol comprising 2 oxyethylene units (CTFA name “Steareth-2”), such as Brij 72, sold by Uniqema; and
the cyclomethicone/dimethicone copolyol mixture sold under the name Q2-3225C® by Dow Corning;
from anionic surfactants:

a) salts of polyoxyethylenated fatty acids, in particular amino salts or alkali metal salts, and their mixtures;

b) salts of C₁₆-C₃₀ fatty acids, in particular amino salts, such as triethanolamine stearate or 2-amino-2-methylpropane-1,3-diol stearate;

c) phosphoric esters and their salts, such as “DEA oleth-10 phosphate” (Crodafos N 10N from Croda) or monopotassium monocetyl phosphate (Amphisol K from Givaudan or Arlatone MAP 160K from Uniqema);

d) sulfosuccinates, such as “Disodium PEG-5 citrate lauryl sulfosuccinate” and “Disodium ricinoleamido MEA sulfosuccinate”;

e) alkyl ether sulfates, such as sodium lauryl ether sulfate;

f) isethionates;

g) acylglutamates, such as “Disodium hydrogenated tallow glutamate” (Amisoft HS-21 R®, sold by Ajinomoto), and their mixtures,
from cationic surfactants:

a) alkylimidazolidiniums, such as isostearylethylimidonium ethosulfate;

b) ammonium salts, such as (C₁₂₋₃₀ alkyl)tri(C₁₋₄ alkyl)ammonium halides, for example N,N,N-trimethyl-1-docosanaminium chloride (or behentrimonium chloride),
from amphoteric surfactants:

N-acylamino acids, such as N-alkylaminoacetates and disodium cocoamphodiacetate, and amine oxides, such as stearamine oxide, or also silicone surfactants, such as dimethicone copolyol phosphates, for example that sold under the name Pecosil PS 100® by Phoenix Chemical.

As specified above, the cosmetic composition (A) and the aqueous composition (B) comprise, in addition to the required compounds described above, a physiologically acceptable medium as defined below.

Physiologically Acceptable Medium

As specified above, the physiologically acceptable medium forming the composition (A) or (B) in accordance with the invention can comprise a fatty phase. The latter can comprise at least one oil and/or at least one wax.

The term “oil” is understood to mean a fatty substance which is liquid at ambient temperature (25° C.) and atmospheric pressure (760 mmHg, i.e. 105 Pa). The oil can be volatile or nonvolatile.

The term “volatile oil” is understood to mean, within the meaning of the invention, an oil capable of evaporating on contact with the skin or with the keratinous fiber in less than one hour, at ambient temperature and atmospheric pressure. The volatile oils of the invention are volatile cosmetic oils which are liquid at ambient temperature and which have a non-zero vapor pressure, at ambient temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

The term “nonvolatile oil” is understood to mean an oil which remains on the skin or the keratinous fiber at ambient temperature and atmospheric pressure for at least several hours and which has in particular a vapor pressure of less than 10⁻³ mmHg (0.13 Pa).

The oil can be chosen from any physiologically acceptable oil and in particular cosmetically acceptable oil, especially mineral, animal, vegetable or synthetic oils; in particular, volatile or nonvolatile hydrocarbon and/or silicone and/or fluorinated oils and their mixtures. More specifically, the term “hydrocarbon oil” is understood to mean an oil comprising mainly carbon and hydrogen atoms and optionally one or more functional groups chosen from hydroxyl, ester, ether or carboxyl functional groups. Generally, the oil exhibits a viscosity of 0.5 to 100 000 mPa·s, preferably of 50 to 50 000 mPa·s and more preferably of 100 to 300 000 mPa·s.

Mention may be made, as example of volatile oil which can be used in the invention, of:
volatile hydrocarbon oils chosen from hydrocarbon oils having from 8 to 16 carbon atoms, in particular branched C₈-C₁₆ alkanes, such as C₈-C₁₆ isoalkanes of petroleum origin (also known as isoparaffins), such as isododecane (also known as 2,2,4,4,6-pentamethylheptane), isodecane or isohexadecane, for example the oils sold under the Isopar® or Permethyl® trade names, branched C₈-C₁₆ esters, isohexyl neopentanoate, and their mixtures. Other volatile hydrocarbon oils, such as petroleum distillates, in particular those sold under the Shell Solt name by Shell, can also be used;
volatile silicones, such as, for example, volatile linear or cyclic silicone oils, in particular those having a viscosity ≦8 centistokes (8×10⁻⁶ m²/s) and having in particular from 2 to 7 silicon atoms, these silicones optionally comprising alkyl or alkoxy groups having from 1 to 10 carbon atoms. Mention may in particular be made, as volatile silicone oil which can be used in the invention, of octamethylcyclotetrasiloxane, decamethylcyclopentasiloxane, dodecamethylcyclohexasiloxane, heptamethylhexyl-trisiloxane, heptamethyloctyltrisiloxane, hexamethyldisiloxane, octamethyltrisiloxane, decamethyltetrasiloxane or dodecamethylpentasiloxane;

and their mixtures.

Mention may also be made of the volatile linear alkyltrisiloxane oils of general formula (I):

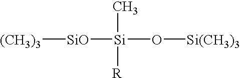

where R represents an alkyl group comprising from 2 to 4 carbon atoms, one or more hydrogen atoms of which can be substituted by a fluorine or chlorine atom.

Mention may be made, among the oils of general formula (I), of:
3-butyl-1,1,1,3,5,5,5-heptamethyltrisiloxane,
3-propyl-1,1,1,3,5,5,5-heptamethyltrisiloxane, and
3-ethyl-1,1,1,3,5,5,5-heptamethyltrisiloxane,

corresponding to the oils of formula (I) for which R is respectively a butyl group, a propyl group or an ethyl group.

Mention may be made, as example of nonvolatile oil which can be used in the invention, of:
hydrocarbon oils of animal origin, such as perhydrosqualene;
vegetable hydrocarbon oils, such as liquid triglycerides of fatty acids of 4 to 24 carbon atoms, such as triglycerides of heptanoic or octanoic acids or also wheat germ, olive, sweet almond, palm, rapeseed, cottonseed, alfalfa, poppy, pumpkinseed, cucumber, blackcurrant, evening primrose, millet, barley, quinoa, rye, safflower, candlenut, passionflower, musk rose, sunflower, corn, soybean, grape seed, sesame, hazelnut, apricot, macadamia, castor or avocado oils, triglycerides of caprylic/capric acids, such as those sold by Stéarineries Dubois or those sold under the names Miglyol® 810, 812 and 818 by Dynamit Nobel, jojoba oil or shea butter;
linear or branched hydrocarbons of mineral or synthetic origin, such as liquid paraffins and their derivatives, liquid petrolatum, polydecenes, polybutenes, hydrogenated polyisobutene, such as Parleam®, or squalane;
synthetic ethers having from 10 to 40 carbon atoms;
synthetic esters, in particular of fatty acids, such as oils of formula R₁COOR₂ in which R₁ represents the residue of a linear or branched higher fatty acid comprising from 1 to 40 carbon atoms and R₂ represents a hydrocarbon chain, in particular a branched hydrocarbon chain, comprising from 1 to 40 carbon atoms with R₁+R₂≧10, such as, for example, purcellin oil (cetearyl octanoate), isononyl isononanoate, isopropyl myristate, isopropyl palmitate, C₁₂ to C₁₅ alkyl benzoate, hexyl laurate, diisopropyl adipate, 2-ethylhexyl palmitate, 2-octyldodecyl stearate, 2-octyldodecyl erucate, isostearyl isostearate or tridecyl trimellitate; octanoates, decanoates or ricinoleates of alcohols or of polyalcohols, such as propylene glycol dioctanoate; hydroxylated esters, such as isostearyl lactate, octyl hydroxystearate, octyldodecyl hydroxystearate, diisostearyl malate, triisocetyl citrate or heptanoates, octanoates or decanoates of fatty alcohols; polyol esters, such as propylene glycol dioctanoate, neopentyl glycol diheptanoate or diethylene glycol diisononanoate; and pentaerythritol esters, such as pentaerythrityl tetraisostearate;
fatty alcohols comprising a branched and/or unsaturated carbon chain having from 12 to 26 carbon atoms which are liquid at ambient temperature, such as octyldodecanol, isostearyl alcohol, 2-butyloctanol, 2-hexyldecanol, 2-undecylpentadecanol or oleyl alcohol;
higher fatty acids, such as oleic acid, linoleic acid or linolenic acid;
carbonates;
acetates;
citrates;
fluorinated oils which optionally to some extent comprise a hydrocarbon and/or silicone part, such as fluorosilicone oils, fluorinated polyethers or fluorinated silicones, such as described in the document EP-A-847 752;
silicone oils, such as polydimethylsiloxanes (PDMSs) which are nonvolatile and linear or cyclic; polydimethylsiloxanes comprising pendant alkyl, alkoxy or phenyl groups or alkyl, alkoxy or phenyl groups at the end of the silicone chain, which groups have from 2 to 24 carbon atoms; phenylated silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates, and
their mixtures.

Thus, the compositions (A) and (B), more particularly the composition (A), can comprise a content of volatile or nonvolatile oils ranging from 0.1 to 80% by weight, with respect to the total weight of the composition, preferably from 0.5 to 70% by weight, preferably from 1 to 50% by weight and more preferably still from 5 to 30% by weight.

Thus, the composition (A) and/or the composition (B), more particularly the composition (A), can also comprise at least one wax.

The wax under consideration in the context of the present invention is generally a lipophilic compound which is solid at ambient temperature (25° C.), which is or is not deformable, which exhibits a reversible solid/liquid change in state and which has a melting point of greater than or equal to 30° C. which can range up to 200° C. and in particular up to 120° C.

On bringing the wax to the liquid state (melting), it is possible to render it miscible with oils and to form a microscopically homogeneous mixture but, on bringing the temperature of the mixture back to ambient temperature, recrystallization of the wax in the oils of the mixture is obtained.

In particular, the waxes suitable for the invention can exhibit a melting point of greater than or equal to 45° C. and in particular of greater than or equal to 55° C.

Within the meaning of the invention, the melting point corresponds to the temperature of the most endothermic peak observed by thermal analysis (abbreviated to DSC) as described in the ISO standard 11357-3; 1999. The melting point of the wax can be measured using a differential scanning calorimeter (DSC), for example the calorimeter sold under the name “MDSC 2920” by TA Instruments.

The measurement protocol is as follows:

A 5 mg sample of wax placed in a crucible is subjected to a first rise in temperature ranging from −20° C. to 100° C. at a heating rate of 10° C./minute, is then cooled from 100° C. to −20° C. at a cooling rate of 10° C./minute and, finally, is subjected to a second rise in temperature ranging from −20° C. to 100° C. at a heating rate of 5° C./minute. During the second rise in temperature, the variation in the difference in power absorbed by the empty crucible and by the crucible comprising the sample of wax is measured as a function of the temperature. The melting point of the compound is the value of the temperature corresponding to the tip of the peak of the curve representing the variation in the difference in power absorbed as a function of the temperature.

The waxes capable of being used in the compositions in accordance with the invention are chosen from waxes of animal, vegetable, mineral or synthetic origin, and their mixtures, which are solid at ambient temperature.

The waxes which can be used in the compositions in accordance with the invention generally exhibit a hardness ranging from 0.01 MPa to 15 MPa, in particular of greater than 0.05 MPa and especially of greater than 0.1 MPa.

The hardness is determined by the measurement of the compressive force measured at 20° C. using a texture analyzer sold under the name TA-XT2 by Rheo, equipped with a stainless steel cylinder with a diameter of 2 mm which is displaced at the measuring rate of 0.1 mm/s and which penetrates the wax to a penetration depth of 0.3 mm.

The measurement protocol is as follows:

The wax is melted at a temperature equal to the melting point of the wax+10° C. The molten wax is cast in a receptacle with a diameter of 25 mm and a depth of 20 mm. The wax is recrystallized at ambient temperature (25° C.) for 24 hours, so that the surface of the wax is flat and smooth, and then the wax is stored at 20° C. for at least one hour before measuring the hardness or the tack.

The rotor of the texture analyzer is displaced at a rate of 0.1 mm/s and then penetrates the wax to a penetration depth of 0.3 mm. When the rotor has penetrated the wax to the depth of 0.3 mm, the rotor is held stationary for 1 second (corresponding to the relaxation time) and is then withdrawn at the rate of 0.5 mm/s.

The value of the hardness is the maximum compressive force measured divided by the surface area of the cylinder of the texture analyzer in contact with the wax.

Mention may in particular be made, by way of illustration of the waxes suitable for the invention, of hydrocarbon waxes, such as beeswax, lanolin wax, Chinese insect waxes, bran wax, rice wax, carnauba wax, candelilla wax, ouricury wax, esparto wax, berry wax, shellac wax, Japan wax, sumac wax, montan wax, orange and lemon waxes, microcrystalline waxes, paraffin waxes and ozokerite, polyethylene waxes, the waxes obtained by the Fischer-Tropsch synthesis and waxy copolymers, and their esters.

Mention may also be made of waxes obtained by catalytic hydrogenation of animal or vegetable oils having linear or branched C₈-C₃₂ fatty chains. Mention may in particular be made, among these, of isomerized jojoba oil, such as the transisomerized partially hydrogenated jojoba oil manufactured or sold by Desert Whale under the commercial reference Iso-Jojoba-50®, hydrogenated sunflower oil, hydrogenated castor oil, hydrogenated coconut oil, hydrogenated lanolin oil and di(1,1,1-trimethylolpropane) tetrastearate, sold under the name of Hest 2T-4S® by Heterene.

Mention may also be made of silicone waxes or fluorinated waxes.

Use may also be made of the waxes obtained by hydrogenation of castor oil esterified with cetyl alcohol which are sold under the names of Phytowax Castor 16L64® and 22L73® by Sophim. Such waxes are described in application FR-A-2 792 190.

According to one embodiment, the composition (A) can comprise at least one wax referred to as a “tacky wax”, that is to say having a tack of greater than or equal to 0.1 N·s and a hardness of less than or equal to 3.5 MPa. The tacky wax used can have in particular a tack ranging from 0.1 N·s to 10 N·s, in particular ranging from 0.1 N·s to 5 N·s, preferably ranging from 0.2 to 5 N·s and better still ranging from 0.3 to 2 N·s.

The tack of the wax is determined by the measurement of the change in the force (compressive force) as a function of the time at 20° C. according to the protocol indicated above for the hardness.

During the relaxation time of 1 s, the force (compressive force) strongly decreases until it becomes zero and then, during the withdrawal of the rotor, the force (stretching force) becomes negative to subsequently again increase towards the value 0. The tack corresponds to the integral of the curve of the force as a function of the time for the part of the curve corresponding to the negative values of the force. The value of the tack is expressed in N·s.

The tacky wax which can be used generally has a hardness of less than or equal to 3.5 MPa, in particular ranging from 0.01 MPa to 3.5 MPa, especially ranging from 0.05 MPa to 3 MPa.

Use may be made, as tacky wax, of a C₂₀-C₄₀ alkyl(hydroxystearyloxy)stearate (the alkyl group comprising from 20 to 40 carbon atoms), alone or as a mixture. Such a wax is sold in particular under the names “Kester Wax K 82 P®”, “Hydroxypolyester K 82 P®” and “Kester Wax K 80 P®” by Koster Keunen.

In the present invention, use may also be made of waxes provided in the form of small particles having a size, expressed as volume-average “effective” diameter D[4,3], of the order of 0.5 to 30 micrometers, in particular of 1 to 20 micrometers and more particularly of 5 to 10 micrometers, subsequently denoted by the expression “microwaxes”.

The sizes of these particles can be measured by various techniques. Mention may in particular be made of light scattering techniques (dynamic and static), Coulter counter methods, measurements by rate of sedimentation (related to the size via Stokes' law) and microscopy. These techniques make it possible to measure a particle diameter and, for some of them, a particle size distribution.

Preferably, the sizes and size distributions of these particles are measured by static light scattering using a commercial particle sizer of MasterSizer 2000 type from Malvern. The data are processed on the basis of the Mie scattering theory. This theory, exact for isotropic particles, makes it possible to determine, in the case of nonspherical particles, an “effective” particle diameter. This theory is described in particular in the work by Van de Hulst, H. C., “Light Scattering by Small Particles”, Chapters 9 and 10, Wiley, New York, 1957.

The composition (A) and/or (B) is characterized by its volume-average “effective” diameter D[4,3], defined in the following way:

$$D [ 4 , 3 ] = \frac{{\sum }_{i} {V}_{i} \cdot {d}_{i}}{{\sum }_{i} {V}_{i}}$$

where Vᵢ represents the volume of these particles with an effective diameter dᵢ. This parameter is described in particular in the technical documentation of the particle sizer.

The measurements are carried out at 25° C. on a dilute dispersion of particles which is obtained from the composition in the following way: 1) diluting by a factor of 100 with water, 2) homogenizing the solution, 3) leaving the solution standing for 18 hours, 4) recovering the off-white homogeneous supernatant.

The “effective” diameter is obtained by taking a refractive index of 1.33 for the water and a mean refractive index of 1.42 for the particles.

Mention may in particular be made, as microwaxes which can be used in the composition (A) and/or (B) in accordance with the invention, of carnauba microwaxes, such as that sold under the name of MicroCare 350® by Micro Powders, synthetic wax microwaxes, such as that sold under the name of MicroEase 114S® by Micro Powders, microwaxes composed of a mixture of carnauba wax and of polyethylene wax, such as those sold under the names of MicroCare 300® and 310® by Micro Powders, microwaxes composed of a mixture of carnauba wax and of synthetic wax, such as that sold under the name MicroCare 325® by Micro Powders, polyethylene microwaxes, such as those sold under the names of Micropoly 200®, 220®, 220L® and 250S® by Micro Powders, and polytetrafluoroethylene microwaxes, such as those sold under the names of Microslip 519® and 519 L® by Micro Powders.

The compositions (A) and/or (B) in accordance with the invention can comprise a content of waxes ranging from 0.1 to 40% by weight, with respect to the total weight of the composition; in particular, it can comprise from 0.5 to 30% by weight thereof.

Structuring Agents

The compositions (A) and/or (B) can comprise structuring agents.

In accordance with the present invention, a structuring agent denotes a compound capable of establishing physical interactions, if appropriate on contact with a crosslinking agent when the structuring agent is not crosslinked, in the fatty phase in which it is employed. It exhibits the ability to develop structuring properties, for example gelling properties, and thus results in textures with a semisolid or solid appearance.

These structuring agents can also be described as “anhydrous gelling agents”, which structure the oils and result in the formation of a solid to semisolid when the gel is allowed to structure on its own account.

Preferably, the agents which structure the oily phase via physical interactions are chosen from the following polymers: polyamides, silicone polyamides, saccharide or polysaccharide mono- or polyalkyl esters, N-acylated amino acid amide derivatives, or copolymers comprising an alkylene or styrene block, it being possible for these copolymers to be diblock, triblock, multiblock or radial-block polymers, the radial-block polymers also being known as star copolymers, or also comb polymers.

The structuring agents can also be polymers which are soluble or dispersible in the oil or oily phase by heating above their melting point M.p. These polymers are in particular block copolymers composed of at least two blocks of different chemical nature, one of which is crystallizable. Reference is made to polymers carrying, in the backbone, at least one crystallizable block suitable for the implementation of the invention; mention may be made of:

1. The polymers defined in the document U.S. Pat. No. 5,156,911;

2. Block copolymers of olefin or of cycloolefin possessing a crystallizable chain, such as those resulting from the block polymerization of:
cyclobutene, cyclohexene, cyclooctene, norbornene (that is to say, bicyclo[2.2.1]hept-2-ene), 5-methylnorbornene, 5-ethylnorbornene, 5,6-dimethylnorbornene, 5,5,6-trimethylnorbornene, 5-ethylidenenorbornene, 5-phenyl-norbornene, 5-benzylnorbornene, 5-vinylnorbornene, 1,4,5,8-dimethano-1,2,3,4,4a,5,8a-octahydronaphthalene, dicyclopentadiene and their mixtures;
with ethylene, propylene, 1-butene, 3-methyl-1-butene, 1-hexene, 4-methyl-1-pentene, 1-octene, 1-decene, 1-eicosene or their mixtures. These block copolymers can in particular be ethylene/norbornene block copolymers and ethylene/propylene/ethylidenenorbornene) block terpolymers.

Use may also be made of those resulting from the block copolymerization of at least two C₂-C₁₆ and better still C₂-C₁₂ α-olefins, such as those mentioned above, and in particular block bipolymers of ethylene and of 1-octene.

The copolymers exhibit at least one crystallizable block, the remainder of the copolymer being amorphous (at ambient temperature). These copolymers can in addition exhibit two crystallizable blocks of different chemical nature. The preferred copolymers are those which simultaneously have, at ambient temperature, a crystallizable block and an amorphous block which is both hydrophobic and lipophilic, these blocks being distributed blockwise; mention may be made, for example, of the polymers having one of the following crystallizable blocks and one of the following amorphous blocks:
block crystallizable by nature: a) polyester, such as poly(alkylene terephthalate)s, b) polyolefin, such as polyethylenes or polypropylenes, and
amorphous and lipophilic block, such as amorphous polyolefins or copoly(olefin)s, such as polyisobutylene, hydrogenated polybutadiene or hydrogenated polyisoprene.

Mention may be made, as examples of such copolymers possessing a crystallizable block and possessing an amorphous block, of:

a) poly(δ-caprolactone)-b-poly(butadiene) block copolymers, used preferably hydrogenated, such as those described in the paper “Melting behavior of poly(δ-caprolactone)-block-polybutadiene copolymers” by S, Nojima, Macromolecules, 32, 3727-3734 (1999),

b) block or multiblock hydrogenated poly(butylene terephthalate)-b-poly(isoprene) block copolymers, cited in the paper “Study of morphological and mechanical properties of PP/PBT” by B. Boutevin et al., Polymer Bulletin, 34, 117-123 (1995),

c) poly(ethylene)-b-copoly(ethylene/propylene) block copolymers, cited in the papers “Morphology of semi-crystalline block copolymers of ethylene-(ethylene-alt-propylene)” by P. Rangarajan et al., Macromolecules, 26, 4640-4645 (1993), and “Polymer aggregates with crystalline cores: the system poly(ethylene)-poly(ethylenepropylene)” by P. Richter et al., Macromolecules, 30, 1053-1068 25 (1997), and

d) poly(ethylene)-b-poly(ethylethylene) block copolymers, cited in the general paper “Crystallization in block copolymers” by I. W. Hamley, Advances in Polymer Science, vol. 148, 113-137 (1999).

The semicrystalline polymers which can be used in the context of the invention may be noncrosslinked or partially crosslinked, provided that the degree of crosslinking does not hinder the dissolution or dispersion in the liquid oily phase by heating above their melting point. Chemical crosslinking may then be involved, by reaction with a polyfunctional monomer during the polymerization. Physical crosslinking may also be involved, which may then be due to either to the establishment of dipolar or hydrogen type bonds between groups carried by the polymer, such as, for example, dipolar interactions between carboxylate ionomers, these interactions being in a small amount and carried by the backbone of a polymer, or to phase separation between the crystallizable blocks and the amorphous blocks carried by the polymer.

Preferably, the semicrystalline polymers suitable for the invention are noncrosslinked.

Mention may be made, as specific example of semicrystalline polymer which can be used in the composition according to the invention, of the Intelimer® products from Landec described in the “Intelimer® Polymers” brochure. These polymers are in their solid form at ambient temperature (25° C.). They carry crystallizable side chains and exhibit the monomer as defined in the preceding formula X. Mention may in particular be made of “Landec IP22®”, having a melting point M.p. of 56° C., which is a product which is viscous at ambient temperature, impermeable and nontacky.

Use may also be made of the semicrystalline polymers described in examples 3, 4, 5, 7 and 9 of the document U.S. Pat. No. 5,156,911, resulting from the copolymerization of acrylic acid and of C₅ to C₁₆ alkyl(meth)acrylate, such as those resulting from the copolymerization:
of acrylic acid, of hexadecyl acrylate and of isodecyl acrylate in a 1/16/3 ratio,
of acrylic acid and of pentadecyl acrylate in a 1/19 ratio,
of acrylic acid, of hexadecyl acrylate and of ethyl acrylate in a 2.5/76.5/20 ratio,
of acrylic acid, of hexadecyl acrylate and of methyl acrylate in a 5/85/10 ratio, and
of acrylic acid and of octadecyl methacrylate in a 2.5/97.5 ratio.

Use may also be made of the polymer “Structure O” sold by National Starch, such as that described in the document U.S. Pat. No. 5,736,125, with an M.p. of 44° C., and also semicrystalline polymers possessing crystallizable pendant chains comprising fluorinated groups, such as described in examples 1, 4, 6, 7 and 8 of the document WO-A-01/19333.

Use may also be made of semicrystalline polymers obtained by copolymerization of stearyl acrylate and of acrylic acid or of NVP or by copolymerization of behenyl acrylate and of acrylic acid or of NVP, such as described in the document U.S. Pat. No. 5,519,063 or EP-A-0 550 745.

According to a specific alternative embodiment, the semicrystalline polymers suitable for the implementation of the present invention are in particular alkyl acrylates, among which may be mentioned the Landec copolymers:
Doresco IPA 13-1®: poly(stearyl acrylate), M.p. of 49° C. and MW of 145 000;
Doresco IPA 13-3®: poly(acrylate/methacrylic acid), M.p. of 65° C. and MW of 114 000;
Doresco IPA 13-4®: poly(acrylate/vinylpyrrolidone), M.p. of 44° C. and MW of 387 000;
Doresco IPA 13-5®: poly(acrylate/hydroxyethyl methacrylate), M.p. of 47° C. and MW of 397 600; and
Doresco IPA 13-6®: poly(behenyl acrylate), M.p. of 66° C.

The compositions (A) and/or (B) can also comprise other structuring agents chosen from thickening agents and gelling agents be chosen from lipophilic gelling agents conventionally used in cosmetics.

Mention may be made, as lipophilic gelling agents conventionally used in cosmetics, for example, of inorganic lipophilic gelling agents, such as clays or silicas, or polymeric organic lipophilic gelling agents, such as organopolysiloxane elastomers which are partially or completely crosslinked, block copolymers of the polystyrene/copoly(ethylene/propylene) type, polyamides and their blends.

With regard to the aqueous phase of the composition (A) and/or (B), it can be thickened by a thickening agent. Mention may be made, among thickening agents for an aqueous phase which can be used according to the invention, of cellulose thickeners, clays, polysaccharides, acrylic polymers, associative polymers and their mixtures.

Mention may in particular be made, as hydrophilic thickening agent, of AMPS/acrylamide copolymers of Sepigel® or Simulgel® type sold by SEPPIC.

In the composition (A) and/or (B) in accordance with the invention, the content of thickening agent for an aqueous phase can range from 0.1% to 15% by weight, with respect to the total weight of the composition, preferably from 1% to 10% by weight and better still from 1% to 5% by weight.

Coloring Materials

The composition (A) and/or the composition (B) in accordance with the invention and more particularly the composition (A) can also comprise at least one coloring material, such as pulverant coloring materials, fat-soluble dyes or water-soluble dyes. This coloring material can be present in a content ranging from 0.1% to 20% by weight, with respect to the total weight of the composition, preferably ranging from 1% to 15% by weight.

Preferably, the aqueous composition (B) is transparent or translucent.

The pulverant coloring materials can be chosen from pigments and pearlescent agents.

The pearlescent agents can be chosen from white pearlescent pigments, such as mica covered with titanium dioxide or bismuth oxychloride, colored pearlescent pigments, such as titanium oxide-coated mica with iron oxides, titanium oxide-coated mica with in particular ferric blue or chromium oxide or titanium oxide-coated mica with an organic pigment of the abovementioned type, and pearlescent pigments based on bismuth oxychloride.

The pigments can be white or colored, inorganic and/or organic and coated or noncoated. Mention may be made, among inorganic pigments, of titanium dioxide, optionally treated at the surface, zirconium, zinc or cerium oxides, and also iron or chromium oxides, manganese violet, ultramarine blue, chromium hydrate and ferric blue. Mention may be made, among organic pigments, of carbon black, pigments of D&C type and lakes, based on cochineal carmine, of barium, strontium, calcium or aluminum.

The fat-soluble dyes are, for example, Sudan Red, D&C Red 17, D&C Green 6, β-carotene, soybean oil, Sudan Brown, D&C Yellow 11, D&C Violet 2, D&C Orange 5, quinoline yellow or annatto. The water-soluble dyes are, for example, beetroot juice or methylene blue.

Fillers

The composition (A) and/or the composition (B) in accordance with the invention and more particularly the composition (A) can comprise at least one filler.

The fillers can be chosen from those well known to a person skilled in the art and commonly used in cosmetic compositions. The fillers can be inorganic or organic and lamellar or spherical. Mention may be made of talc, mica, silica, kaolin, powders formed of polyamide, such as Nylon® (Orgasol® from Atofina), of poly-β-alanine and of polyethylene, powders formed of tetrafluoroethylene polymers, such as Teflon®, lauroyllysine, starch, boron nitride, hollow polymer microspheres which are expanded, such as those of poly(vinylidene chloride)/acrylonitrile, for example Expancel® (Nobel Industrie), acrylic powders, such as Polytrap® (Dow Corning), particles formed of polymethyl methacrylate and silicone resin microbeads (Tospearls® from Toshiba, for example), precipitated calcium carbonate, magnesium carbonate, basic magnesium carbonate, hydroxyapatite, hollow silica microspheres (Silica Beads® from Maprecos), glass or ceramic microcapsules, or metal soaps derived from organic carboxylic acids having from 8 to 22 carbon atoms, preferably from 12 to 18 carbon atoms, for example zinc stearate, magnesium stearate, lithium stearate, zinc laurate or magnesium myristate.

The fillers can represent from 0.1 to 25% by weight and better still from 1 to 20% by weight of the total weight of the composition in which they occur.

Other Additives

The composition (A) and/or (B) in accordance with the invention can additionally comprise any additive conventionally used in cosmetics, such as antioxidants, preservatives, fragrances, neutralizing agents, plasticizing agents, cosmetic active principles, such as, for example, emollients, moisturizing agents, vitamins or sunscreens, and their mixtures. These additives can be present in the composition in a content ranging from 0.01 to 30% of the total weight of the composition.

Of course, a person skilled in the art will take care to choose the optional additional additive(s) and/or their amounts so that the advantageous properties of said cosmetic composition (A) and aqueous composition (B) are not, or not substantially, detrimentally affected by the envisaged addition.

According to a preferred alternative embodiment, the incorporation of these additives is favored in the composition (A).

Makeup Kit

As specified above, the kit according to the present invention comprises (i) at least one cosmetic composition (A) and (ii) water or more particularly an aqueous composition (B). It can in addition advantageously comprise one or more means for applying said compositions to the surface to be made up.

The cosmetic composition (A) and the water or the cosmetic composition (A) and the aqueous composition (B) included in the makeup kit of the invention can in particular be applied to the eyelashes using a brush or a comb.

The thickening effect on the makeup can furthermore be reinforced by very particularly selecting the means for applying the cosmetic composition (A) or the cosmetic composition (A) and the aqueous composition (B), which can in particular be a makeup brush.

As it happens, it is particularly advantageous, in the case of making up the eyelashes, to apply at least one of said compositions with a makeup brush as described in patents FR 2 701 198, FR 2 605 505, EP 792 603 and EP 663 161.

The make up kit according to the invention can, according to a specific embodiment, comprise at least two separate packagings, one comprising the cosmetic composition (A) as defined above and the other comprising the water or the aqueous composition (B) also defined above.

When the kit is in the form of one and the same packaging, it can be provided as a container delimiting at least one compartment or reservoir which comprises one of the two compositions (A) or (B), said compartment being closed by a closing element, and at least one compartment or reservoir which comprises the other composition, also being closed by a closing element.

In this embodiment, the kit is preferably used in combination with at least one application means or applicator, in particular in the form of a brush comprising an arrangement of hairs held by a twisted wire. Such a twisted brush is described in particular in patent U.S. Pat. No. 4,887,622. It can also be in the form of a comb comprising a plurality of application elements, obtained in particular from molding. Such combs are described, for example, in patent FR 2 796 529. The applicator can be integrally attached to the container, as described, for example, in patent FR 2 761 959. Advantageously, the applicator is integrally attached to a rod which is itself integrally attached to the closing element.

The closing element can be coupled to the container by screwing. Alternatively, the coupling between the closing element and the container is carried out other than by screwing, in particular via a bayonet mechanism, by snapping or by clamping. The term “snapping” is understood to mean in particular any system involving the crossing of a row or strip of material by elastic deformation of a portion, in particular of the closing element, and then by elastically returning said portion to the unstressed position after the row or strip has been crossed.

The container, advantageously comprising two compartments or reservoirs, can be at least partially made of thermoplastic material. Mention may be made, as examples of thermoplastic materials, of polypropylene or polyethylene.

Alternatively, the container is made of nonthermoplastic material, in particular of glass or of metal (or alloy).

The container is preferably equipped with a drainer positioned in the vicinity of at least one opening of the container. Such a drainer makes it possible to wipe the applicator and optionally the rod to which it may be integrally attached. Such a drainer is described, for example, in patent FR 2 792 618.

The contents of the abovementioned patents or patent applications are incorporated by way of reference in the present patent application.

According to a particularly preferred embodiment, the makeup kit comprises two reservoirs, one dedicated to the composition (A) and the other to the composition (B), each of the reservoirs being provided with a makeup brush, in particular of mascara brush type as described above.

The examples and figures which follow are presented by way of illustration and without implied limitation of the invention.

### EXAMPLE 1

### Mascara Composition (A)

| Ingredients | % by weight |
|---|---|
| Styrene/acrylate/ammonium methacrylate copolymer as | 60 |
| an aqueous dispersion comprising 40% of active material | |
| (Syntran ® 5760 from Interpolymer) | |
| Acrylamide/Na AMPS copolymer in isohexadecane with | 4.5 |
| Polysorbate 80 (Simulgel 600 ® from SEPPIC) | |
| Propylene glycol | 8 |
| Black iron oxide | 7 |
| Preservatives | q.s. |
| Water | q.s. for 100 |

The effectiveness of this composition (A) was tested on test specimens of false eyelashes, in order to obtain an artificial elongation of these false eyelashes in accordance with the invention, according to the following protocol.

Test specimens of false eyelashes numbered 1, 5, 6, 7, 9 and 10 are prepared with straight black Caucasian hairs with a fringe length of 19 mm. The fringes of these false eyelashes are mounted between two 30 mm by 30 mm plates which embody the eyelid.

The false eyelashes of the test specimens prepared above were treated according to the following process:
Stage 1: 30 passes over the false eyelashes of a mascara brush with the composition defined above possessing an aqueous continuous phase,
Stage 2: drying the mascara thus deposited on the false eyelashes,
Stage 3: 20 passes over false eyelashes made-up above of a mascara brush impregnated with water,
Stage 4: for the test specimens 5, 6, 7, 9 and 10, displacement of the moistened sheaths of composition (A) using tweezers which simultaneously grasp all the sheaths, the jaws of the tweezers being positioned perpendicularly to the false eyelashes, and traction of the sheaths in the direction of the upper end of the false eyelashes, parallel to the false eyelashes.

For the test specimen 1, this displacement stage 4 is not applied to the sheaths of the false eyelashes. In this case, direct progression occurs from the preceding moistening stage (stage 3) to the following drying stage (stage 5).
Stage 5: subsequent drying of the sheaths, which become integrally attached to the false eyelashes with which they are associated.

On conclusion of stage 5, the test specimens 1, 5, 6, 7, 9 and 10 are photographed (see FIG. 2).

Results:

On conclusion of the above process, the mean lengths of the false eyelashes are measured for each of the test specimens 1, 5, 6, 7, 9 and 10 and the elongation of the false eyelashes resulting from the displacement of the mascara sheaths is calculated using, as reference, the length of the false eyelashes of test specimen 1. The results are combined in table 1 below.

TABLE 1

| | Measured mean length of | Calculated mean |
|---|---|---|
| | the made-up false | elongation of the made- |
| Test specimens | eyelashes (cm) | up false eyelashes (cm) |
| 1 | 14 | 0 |
| 5 | 16 | 2 |
| 6 | 18 | 4 |
| 7 | 21 | 7 |
| 9 | 17 | 3 |
| 10 | 18 | 4 |

### EXAMPLE 2

### Mascara Composition (A)

| Ingredients | % by weight |
|---|---|
| Aliphatic polyurethane as an aqueous dispersion | 60 |
| comprising 38% of active material (Avalure UR 450 ® | |
| from Novéon) | |
| Acrylamide/Na AMPS copolymer in isohexadecane with | 4.5 |
| Polysorbate 80 (Simulgel 600 ® from SEPPIC) | |
| Propylene glycol | 8 |
| Black iron oxide | 7 |
| Preservatives | q.s. |
| Water | q.s. for 100 |

1. A method for making up the eyelashes which comprises at least the following stages:
a) forming, over all or part of the eyelashes, cohesive sheaths resulting from the application of at least one layer of at least one cosmetic composition (A) comprising at least 15% by weight, expressed as weight of dry matter, with respect to the total weight of said composition, of at least one film-forming compound, and
b) displacing the sheaths towards the upper end of the eyelashes via a translational movement along the eyelashes over a distance which is less than the length of the eyelashes.

2. The method as claimed in claim 1, wherein the sheaths are displaced over a distance of less than three quarters of the length of the non-made-up eyelashes.

3. The method as claimed in claim 1, wherein the sheaths around the eyelashes are displaced so as to provide an elongation of at least 10%, in particular of at least 20%, with respect to the original length of the non-made-up eyelashes.

4. The method as claimed in claim 1, wherein the displacement of the sheaths is facilitated by bringing the latter into contact with a composition (B) capable of reducing adhesion of the sheaths to the eyelashes.

5. The method as claimed in claim 4, wherein the composition (B) intended to reduce the adhesion of the sheaths to the eyelashes is applied subsequently to the formation of the sheaths.

6. The method for making up the eyelashes as claimed in claim 1 which comprises at least the following stages:
a) applying, at the surface of the eyelashes, at least one layer of at least one cosmetic composition (A) comprising at least 15% by weight, of dry matter, with respect to the total weight of said composition, of at least one film-forming compound,
b) allowing the cosmetic composition (A) applied in a) to dry in order to form sheaths around the eyelashes,
c) bringing the sheaths of composition (A), formed in stage b), into contact with pure water or an aqueous composition (B), and
d) displacing the sheaths, thus moistened, towards the upper end of the eyelashes via a translational movement along the eyelashes over a distance which is less than the length of the eyelashes.

7. The method as claimed in claim 6, wherein the composition (A) in stage c) is brought into contact with an amount of pure water or of aqueous composition (B) sufficient to bring about swelling of the sheath or sheaths of cosmetic composition (A).

8. The method as claimed in claim 1, wherein the film-forming compound is a polymer obtained by polymerization or copolymerization of monomers chosen from styrene, butadiene, acrylonitrile, chloroprene, vinyl acetate, urethanes, isoprene, isobutylene and acrylic or methacrylic, maleic, crotonic or itaconic acids or their esters or amides or by dispersion of a polymer in an aqueous phase.

9. The method as claimed in claim 1, wherein the film-forming compound is a polymer in the form of particles in dispersion in an aqueous phase.

10. The method as claimed in claim 1, wherein the film-forming compound is a polymer chosen from synthetic polymers of the polycondensate type or of the radical type, and the blends of these.

11. The method as claimed in claim 10, wherein the synthetic polymers of polycondensate type are chosen from polyurethanes, which may be anionic, cationic, nonionic or amphoteric, polyurethane-acrylics, polyurethane-polyvinylpyrrolidones, polyester-polyurethanes, polyether-polyurethanes, polyureas, polyurea-polyurethanes, and blends of these, or also from polyesters, polyesteramides, fatty-chain polyesters, polyamides and epoxy ester resins.

12. The method as claimed in claim 10, wherein the radical polymers are acrylic and/or vinyl homopolymers or copolymers, acrylic/silicone copolymers or nitrocellulose/acrylic copolymers or also hybrid polymers.

13. The method as claimed in claim 1, wherein the film-forming compound is a polymer chosen from polyurethanes.

14. The method as claimed in claim 1, wherein cosmetic composition (A) comprises from 15 to 60% by weight of dry matter of film-forming compound(s), with respect to the total weight of said composition, preferably from 20 to 50% by weight and more advantageously still from 22 to 40% by weight.

15. The method as claimed in claim 1, wherein the cosmetic composition (A) additionally comprises at least one polyelectrolyte.

16. The method as claimed in claim 15, wherein the polyelectrolyte is chosen from acrylamide/2-methyl-2-[(1-oxo-2-propenyl)amino]-1-propanesulfonic acid copolymers, a crosslinked starch glycolate in the powder form, a polyacrylate, a grafted starch-based copolymer, ionizable derivatives of polysaccharides, polyacrylic acids, polyacrylic acid/alkyl acrylate copolymers, AMPS (polyacrylamidomethylpropanesulfonic acid partially neutralized with ammonia and highly crosslinked), copolymers of AMPS and of alkyl methacrylates which are polyoxyethylenated (and crosslinked or noncrosslinked), ionizable derivatives of cellulose, and their mixtures.

17. The method as claimed in claim 15, wherein the polyelectrolyte is chosen from an acrylamide/2-methyl-2-[(1-oxo-2-propenyl)amino]-1-propanesulfonic acid copolymer, a polyacrylate and their copolymers.

18. The method as claimed in claim 15, wherein the polyelectrolyte is present in a content as dry matter ranging from 0.05 to 15% by weight, more preferably still from 0.1 to 10% by weight and better still from 0.5 to 5% by weight, with respect to the total weight of the cosmetic composition (A).

19. The method as claimed in claim 6, wherein in stage c), the sheaths of composition (A) formed in stage b) are brought into contact with an aqueous composition (B).

20. The method as claimed in claim 6, wherein in stage c), the sheaths of composition (A) formed in stage b) are brought into contact with pure water.

21. The method as claimed in claim 4, wherein the aqueous composition (B) comprises at least one surfactant.

22. The method as claimed in claim 4, wherein the aqueous composition (B) is transparent or translucent.

23. The method as claimed in claim 1, wherein the cosmetic composition (A) comprises an aqueous continuous phase.

24. The method as claimed in claim 1, wherein the cosmetic composition (A) comprises an oily or solvent continuous phase.

25. The method as claimed in claim 1, wherein the cosmetic composition (A) comprises at least one coloring material and/or at least one filler.

26. The method as claimed in claim 1, wherein the cosmetic composition (A) comprises at least one wax.

27. The method as claimed in claim 1, wherein the cosmetic composition (A) is provided in the form of a mascara.

28. A kit for making up the eyelashes, comprising, in separate fashion, at least one cosmetic composition (A) comprising at least 15% by weight, expressed as dry matter, with respect to the total weight of the composition, of at least one film-forming compound and an aqueous composition (B) other than (A).

29. The kit as claimed in claim 28, wherein the film-forming compound is a polymer obtained by polymerization or copolymerization of monomers chosen from styrene, butadiene, acrylonitrile, chloroprene, vinyl acetate, urethanes, isoprene, isobutylene and acrylic or methacrylic, maleic, crotonic or itaconic acids or their esters or amides or by dispersion of a polymer in an aqueous phase.

30. The kit as claimed in claim 28, wherein the polyelectrolyte is chosen from acrylamide/2-methyl-2-[(1-oxo-2-propenyl)amino]-1-propanesulfonic acid copolymers, a crosslinked starch glycolate in the powder form, a polyacrylate, a grafted starch-based copolymer, ionizable derivatives of polysaccharides, polyacrylic acids, polyacrylic acid/alkyl acrylate copolymers, AMPS (polyacrylamidomethylpropanesulfonic acid partially neutralized with ammonia and highly crosslinked), copolymers of AMPS and of alkyl methacrylates which are polyoxyethylenated (and crosslinked or noncrosslinked), ionizable derivatives of cellulose, and their mixtures.

31. The kit as claimed in claim 28, further comprising application means dedicated to the application of each of the compositions.

32. The kit as claimed in claim 28, wherein it comprises at least one brush or comb dedicated to the application of the composition (A).

33. The kit as claimed in claim 28, wherein it comprises a device favorable to the displacement of the sheaths of cosmetic composition (A) towards the upper ends of said eyelashes.

34. The kit as claimed in claim 28, wherein it comprises several compositions (A) which are different from one another and which are dedicated to providing different makeups in terms of color and/or optical effect.

35. The kit as claimed in claim 28, wherein the cosmetic composition (A) is a mascara.

36. (canceled)
